(19) United States
(12) Patent Application Publication
(10) Pub. No.: US 2010/0129476 A1
(43) Pub. Date: May 27, 2010

(54) COMPOSITION COMPRISING REHMANNIAE RADIX PREPARATA, NOTOGINSENG RADIX OR MIXTURE EXTRACT THEREOF FOR PREVENTING AND TREATING OF PERIODONTITIS AS AN EFFECTIVE COMPONENT

(75) Inventors: Jung-Keun Kim, Seongnam-si (KR); Se-Won Kim, Cheonan-si (KR); Dong-Heon Baek, Seoul (KR); Hyung-Gun Kim, Seoul (KR)

Correspondence Address: SUGHRUE MION, PLLC, 2100 PENNSYLVANIA AVENUE, N.W., SUITE 800, WASHINGTON, DC, 20037, US

(73) Assignee: OSCOTEC INC., Cheonan-si, Chungcheongnam-do (KR)

(21) Appl. No.: 12/596,014

(22) PCT Filed: Apr. 4, 2008
(86) PCT No.: PCT/KR2008/001924

§ 371 Date: Oct. 15, 2009

(30) Foreign Application Priority Data
Apr. 18, 2007 (KR) 10-2007-0037997

(51) Int. Cl. A61K 36/258; A61K 36/00; A61P 1/02

(52) U.S. Cl. 424/728; 424/725

(57) ABSTRACT

Disclosed is a pharmaceutical composition for the treatment and prevention of periodontal diseases, comprising an extract from Rehmanniae Radix Preparata, Panax notoginseng (Burk.) F. H. Chen or a mixture thereof as an active ingredient. Having the activity of protecting the alveolar bone and promoting the proliferation of periodontal ligament cells as well as inhibiting the release of TNF-α, the extract can be applied to the preparation of a pharmaceutical composition or a health food composition useful in the treatment and prevention of periodontal diseases.

### TECHNICAL FIELD

The present invention relates to a composition for the prevention and treatment of periodontitis, comprising an extract from Rehmanniae Radix Preparata, Notoginseng Radix or a mixture thereof as an active ingredient.

### BACKGROUND ART

The periodontal tissue may be divided into the alveolar bone, the gingiva and the periodontal ligament. The gingiva, a structure supporting the teeth, can be affected with gingivitis. When spreading over the supporting structures of teeth to destroy the periodontal ligament, which attaches the cementum of a tooth to the alveolar bone and covers the root of the tooth within the bone, and the alveolar bone, which forms the alveolus around teeth, inflammation leads to periodontitis.

Periodontal diseases including gingivitis and periodontitis refer to the inflammation of tooth-supporting structures, caused by bacteria, and are accompanied by hemorrhage, the formation of periodontal pockets, and the destruction of the alveolar bone, resulting in the loss of teeth. Such periodontitis is developed in the process of the formation of bacterial colonies, the infiltration of bacteria into periodontal tissues, and the destruction of the periodontal tissues. In detail, a bacterial biofilm formed under conditions of poor oral sanitation may cause inflammation in the gums with the accompaniment of hemorrhage and halitosis. The progression of this oral state develops a gap between a tooth and the gingiva to form a periodontal pocket where bacteria then proliferate with the outbreak of periodontitis. When deteriorating, periodontitis makes the gums bleed even upon a weak stimulus such as tooth brushing and often develops into acute inflammation with the occurrence of pain. This inflammation lowers osteogenetic function and enhances bone resorption to reduce the alveolar bone, resulting in the loss of teeth.

There are many causes of periodontitis. Locally, when plaque and calculus accumulates within a periodontal pocket, it acts as habitat for neighboring anaerobic gram negative bacteria which proliferate to the deep core of the periodontal pocket. The toxins and products of the proliferated anaerobic gram negative bacteria directly destroy periodontal tissues or stimulate the immune system, which then induces the destruction of the periodontal tissue with the accompaniment of inflammation. For a defense mechanism against the destruction, multinucleate leukocytes and immune responses act as systemic factors.

As a result of the metabolism of anaerobic gram bacteria, the bacterial toxins hydrogen sulfide, ammonia and amines, which are toxic to the periodontal tissue, are secreted while the endotoxin lipopolysaccharide, which is a constituent of the cell wall, directly destroys the periodontal tissue or stimulates the immune system. Various actions of stimulated humoral and cellular immunity induce the extracellular secretion of active oxygen, prostaglandins, leukotrienes, histamine, and cytokines such as interleukins, incurring periodontal inflammation. Also, the collagenase secreted from bacteria and leukocytes digests collagen, the matrix of the periodontal tissue, to give rise to gingival degeneration, which leads to periodontal diseases.

Therefore, it is the key for the prevention and treatment of periodontal diseases to kill or inhibit anaerobic gram negative bacteria, remove the toxic products from the bacteria, or recover the degenerated periodontal tissue.

For the treatment of periodontal diseases, an improvement in oral sanitation, or non-surgical or surgical therapies including periodontal regeneration such as scaling, root planing, gingival curettage and new attachment, have been used. However, such surgical therapies require the patients to go to dental clinics. In addition, the surgical therapies are applied to the treatment of cases of periodontal diseases that have progressed to some degree, in most cases, to a chronic state, rather than the prevention thereof, and are accompanied with antibiotics for systemic application or sustained-release agents for local application. The drugs may be delivered in too great an amount to unnecessary parts to avoid side effects thereof. In fact, it has been reported that periodontitis bacteria resistant to antibiotics have been isolated.

Therefore, there is a need for an agent or composition for the prevention and treatment of periodontal diseases that has the activity of reestablishing destroyed or lost periodontal tissues.

According to Melcher, the result of periodontal regeneration varies depending on the origin of the cells involved in periodontal regeneration. For example, the result includes osseointegration when the cells originating from the bone are involved in regeneration, root resorption when the cells originating from the connective tissue (e.g. gingival fibroblast) are involved in regeneration, and the generation of long attachment epithelium when cells originating from the epithelial tissue are involved in regeneration. However, these results are different from the ideal regeneration of periodontal tissues. In contrast, ideal regeneration of periodontal tissues can be induced when the cells originating from periodontal ligament cells are involved in regeneration (Melcher A. H. Journal of Periodontology, 47(5); 256-260, 1976).

In brief, it is ideal to the regeneration of periodontal tissues to promote the growth of periodontal ligament cells without affecting the growth of gingival fibroblast cells.

Based on the fact that when they are activated, CD4⁺ T cells, which induce the secretion of various kinds of cytokines (TNF-α, IFN-γ, GM-CSF, IL-2, IL-6) which are secreted by the immune system upon stimulation with bacterial toxins, express osteoprotegerin ligands on the surface thereof to promote osteoclastogenesis, thereby playing a critical role in the destruction of bone, the treatment of periodontal diseases, in the opinions of the present inventors, requires suppressing cytokine release in order to prevent inflammation and the destruction of the alveolar bone (Kong Y. Y. et al., Nature 402, 304-309, 1999), promoting the proliferation and differentiation of osteoblast cells and reducing the formation and activity of osteoclast cells to protect the alveolar bone, and effectively increasing the proliferation of periodontal ligament cells without affecting the proliferation of gingival fibroblast cells.

Leading to the present invention, intensive and thorough research into various physiological activities of Rehmanniae Radix Preparata, Notoginseng Radix, conducted by the present inventors, resulted in the finding that an extract from Rehmanniae Radix Preparata, Notoginseng Radix or a mixture thereof has the functions of suppressing the release of cytokines responsible for inflammation to prevent inflammation, promoting the proliferation and differentiation of osteoblast cells and reducing the formation and activity of osteoclast cells to protect the alveolar bone, and effectively increasing the proliferation of periodontal ligament cells without affecting the proliferation of gingival fibroblast cells, and thus can be useful in the prevention and treatment of periodontal diseases.

### DISCLOSURE

### Technical Problem

It is therefore an object of the present invention to provide a composition for the treatment and prevention of periodontal diseases, comprising a mixture of respective extracts from Rehmanniae Radix Preparata and Notoginseng Radix as an active ingredient for suppressing inflammation attributed to periodontal diseases, promoting the proliferation and differentiation of osteoblast cells, reducing the formation and activity of osteoclast cells to prevent the destruction of the alveolar bone, and stimulating the proliferation of periodontal ligament cells.

### Technical Solution

In order to accomplish the above object, the present invention provides a pharmaceutical composition for the treatment and prevention of a periodontal disease, comprising one of respective extracts from Rehmanniae Radix Preparata and Panax notoginseng (Burk.) F. H. Chen, a mixture of the respective extracts, or an extract from a mixture of Rehmanniae Radix Preparata and Panax notoginseng (Burk.) F. H. Chen as an active ingredient.

Also, the present invention provides a health food composition for the treatment and prevention of a periodontal disease, comprising one of respective extracts from Rehmanniae Radix Preparata and Panax notoginseng (Burk.) F. H. Chen, a mixture of the respective extracts, or an extract from a mixture of Rehmanniae Radix Preparata and Panax notoginseng (Burk.) F. H. Chen as an active ingredient.

### ADVANTAGEOUS EFFECTS

Having the activity of protecting the alveolar bone and promoting the proliferation of periodontal ligament cells as well as inhibiting the release of TNF-α, the extract according to the present invention is applicable to the treatment and prevention of periodontal diseases.

### DESCRIPTION OF DRAWINGS

FIG. 1 is a graph showing the inhibitory effect of the extracts of the present invention on the release of TNF-α according to the mixture ratio of the extracts.

FIG. 2 is a graph showing the inhibitory effect of the herbal mixture extracts of the present invention on the release of TNF-α.

FIG. 3 is a graph showing the effect of the extracts of the present invention on the proliferation of osteoblast cells according to the mixture ratio of the extracts.

FIG. 4 is a graph showing the effect of the extracts of the present invention on the expression of osteoprotegerin (OPG) in osteoblast cells according to the mixture ratio of the extracts.

FIG. 5 is a graph showing the effect of the herbal mixture extract of the present invention on the expression of OPG in osteoblast cells.

FIG. 6 is a graph showing the effect of the extracts of the present invention on the formation of osteoclast cells according to the mixture ratio of the extracts.

FIG. 7 is a graph showing the effect of the herbal mixture extract of the present invention on the formation of osteoclastic cells.

FIG. 8 is a graph showing the effect of the extracts of the present invention on the activity of osteoclast cells according to the mixture ratio of the extracts.

FIG. 9 is a graph showing the effect of the herbal mixture extract of the present invention on the activity of osteoclast cells.

FIG. 10 is a graph showing the effect of the extracts of the present invention on the proliferation of human gingival fibroblasts according to the mixture ratio of the extracts.

FIG. 11 is a graph showing the effect of the herbal mixture extract of the present invention on the proliferation of human gingival fibroblasts.

FIG. 12 is a graph showing the effect of the extracts of the present invention on the proliferation of human periodontal ligament cells according to the mixture ratio of the extracts.

FIG. 13 provides histochemical photographs showing the inhibitory effect of the herbal mixture extract of the present invention on bone loss in rats afflicted with periodontitis (AC and CEJ respectively represent Alveolar bone Crest and Cemento-Enamel Junction).

FIG. 14 is a graph showing the inhibitory effect of the herbal mixture extract of the present invention on bone loss in rats afflicted with periodontitis.

### BEST MODE

Hereinafter, embodiments of the present invention will be described in detail.

The present invention pertains to a composition for the treatment and prevention of periodontal diseases, comprising a mixture of extracts from Rehmanniae Radix Preparata and Notoginseng Radix.

In accordance with the present invention, a mixture of 1:8 Rehmanniae Radix Preparata: Notoginseng Radix is subjected three times to extraction with hot water and the solution is filtered and concentrated in a vacuum at 40° C. or lower to afford an extract mixture useful for the prevention and treatment of periodontal diseases.

Rehmanniae Radix Preparata is an herb belonging the Scrophulariaceae family and its fresh or dry roots are used as a medicinal material in the herb medicine. Particularly, the roots obtained after nine rounds of steaming and drying have medicinally excellent efficacy. Having a warm character, the roots that taste sweet and bitter act to nourish the blood and supplement the essence. In the herb medicine, thus, the roots are applied the treatment of pains in the waist and the knee, menstrual pain and vertigo. Also, they are known to function to prevent hair decolorization. Rehmanniae Radix Preparata is a main ingredient of a decoction of the four herbs. It is used for the treatment of a fever attributed to a weak body constitution, dryness in the throat, and thirst. In Korean folk remedies, the herb is eaten along with a pork soup to treat chronic constipation.

Notoginseng Radix (Panax notoginseng (Burk.) F. H. Chen) is a perennial herb belonging to the ginseng family, which is smaller than ginseng, has seven leaves and is widely cultured in Southern regions of China. Its roots look like a small shuttle. The herb is also called 3-7 ginseng because it has three stems with 7 leaves attached thereto and looks like a ginseng. Its roots with a saponin content of 3-8% contains ginsenosides Rb1, Rg1, and Re and notoginsenosides R1, R2, Fa and Fc in large amounts, and ginsenosides R2, b2, d, e and c in small amounts and ginsenoside R0 in a trace amount. Its essential oils are smaller in number than ginseng and include oleanolic acid. The roots have been in the herbal medicine used for hemostasis and as cardiotonic agents and were found to have functions of increasing the blood flow through the cardiac artery, decreasing the oxygen consumption of the cardiac muscle and reducing the levels of lipid and cholesterol in the blood, as measured in animal experiments. Having excellent anti-inflammatory, analgesic and hemostatic activities, Notoginseng Radix is also useful for the treatment not only of inflammatory diseases including hepatitis, but also of external or internal hemorrhaging through direct application to wounds or oral administration.

The extract from Rehmanniae Radix Preparata, Notoginseng Radix or a mixture thereof can be prepared through a conventional method such as cold precipitation, hot precipitation, heating, etc. Preferably, Rehmanniae Radix Preparata, Notoginseng Radix or a mixture thereof is treated with water, alcohol or a mixture thereof. 8 weight parts of Notoginseng is preferably used per weight part of Rehmanniae Radix Preparata. The alcohol is preferably methanol or ethanol. More preferably, Rehmanniae Radix Preparata, Notoginseng Radix or a mixture thereof is immersed in methanol or ethanol at 70-80° C. for 3-5 hrs. The repetition of this procedure may increase the efficiency of extraction. The concentration may be carried out under a condition of 20-40° C. in a vacuum, but is not limited thereto. As for the individual extracts from Rehmanniae Radix Preparata or Notoginseng Radix, they may be mixed with each other in a weight ratio of 1-16:16-1.

The main cytokines responsible for bone loss, inflammation and connective tissue destruction associated with periodontal diseases are known to be interleukin-1 (IL-1) and tumor necrosis factor (D. T. Graves and D. Cochran, The contribution of Interleukin-1 and Tumor Necrosis Factor to Periodontal Tissue Destruction, Journal of Periodontology, 74(3):391-401, 2003).

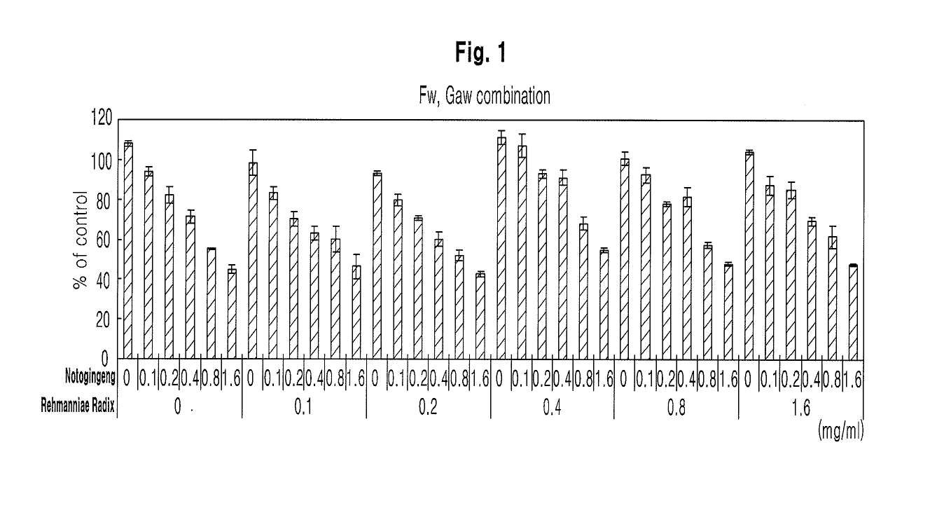

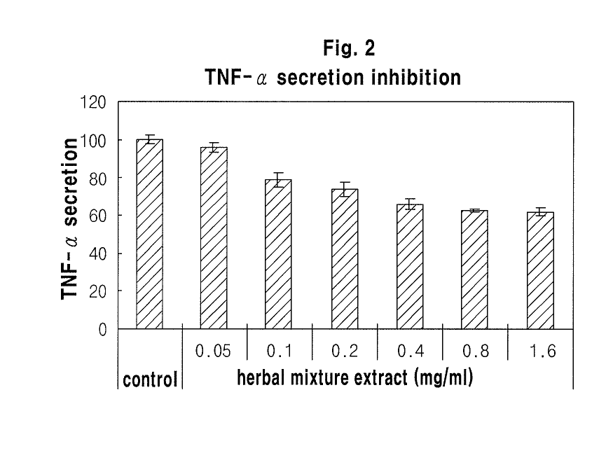

An examination was made of the effect of the extract according to the present invention on the secretion of tumor necrosis factor-α (hereinafter referred to as “TNF-α”). In this regard, the human monocytic cell line THP-1 was treated with lipopolysaccharide (LPS) and the extract according to the present invention, followed by analyzing the level of TNF-α in the cell medium through enzyme-linked immunosorbent assay (ELISA). As a result, the secretion of TNF-α was found to be significantly suppressed upon treatment with 0.1 mg/ml of the extract according to the present invention (FIGS. 1 and 2). Hence, the extract according to the present invention can be used as a suppressant against TNF-α secretion.

The osteogenesis of the alveolar bone is known to increase with increasing OPG secretion (M. A. Taubman, P. Valverde, X. Han and T. Kawai, Immune Response: The Key to Bone Resorption in Periodontal Disease, Journal of Periodontology, 76(11 Suppl):2033-41, 2005).

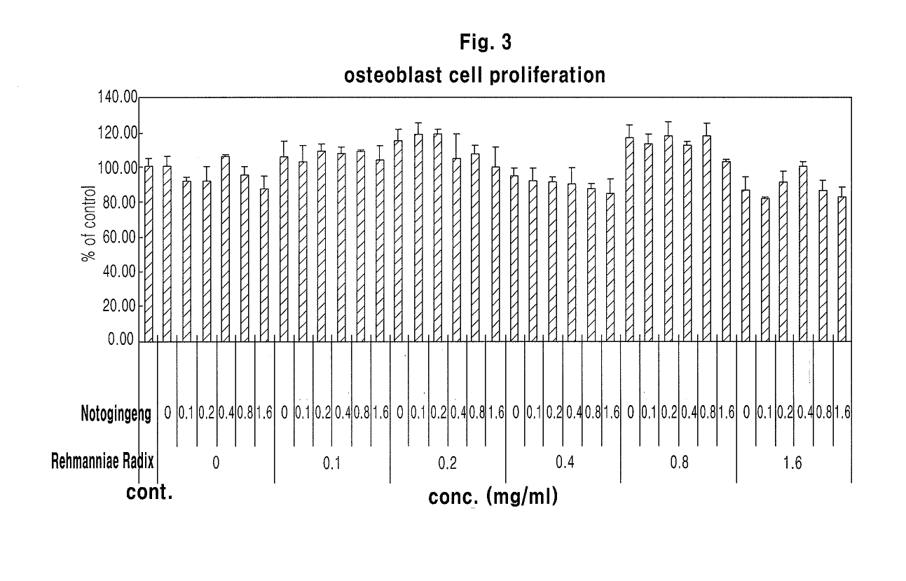

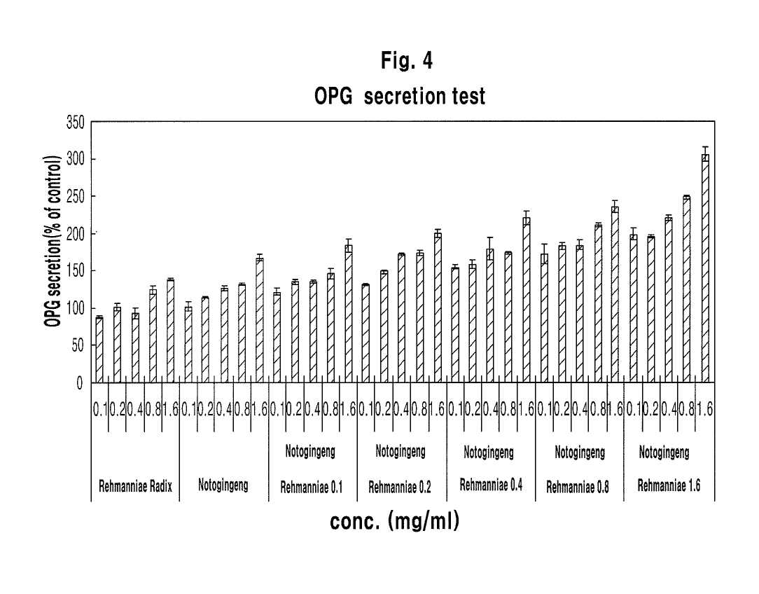

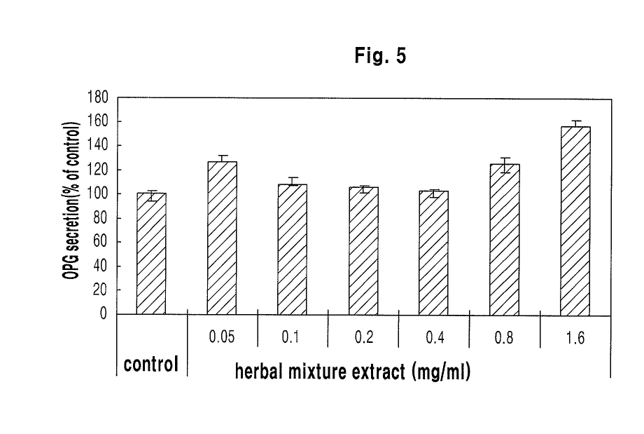

When osteoblast cells were treated with the extract according to the present invention, they proliferated increasingly as the concentration of the extract was increased (FIG. 3). Also, human osteosarcoma MG-63, when treated with the extract according to the present invention, secreted OPG in a dose-dependent manner (FIGS. 4 and 5).

Osteoclast cells are generated from monocytes/macrophage progenitors in the bone marrow, and the monocytic progenitor cells are circulated through blood, proliferate in the endosteal layer and fuse with each other to form multinuclear cells (Scheven, B. A. A. et al., Nature, 321: 79-81, 1986). Osteoclasts are characterized by their expression of tartrate-resistant acidic phosphatase (TRAP), which is usually used as a histochemical enzyme for discriminating osteoclasts from other bone tissue cells (Minkin, C., Calcif. Tissue Int., 34: 285-290, 1982).

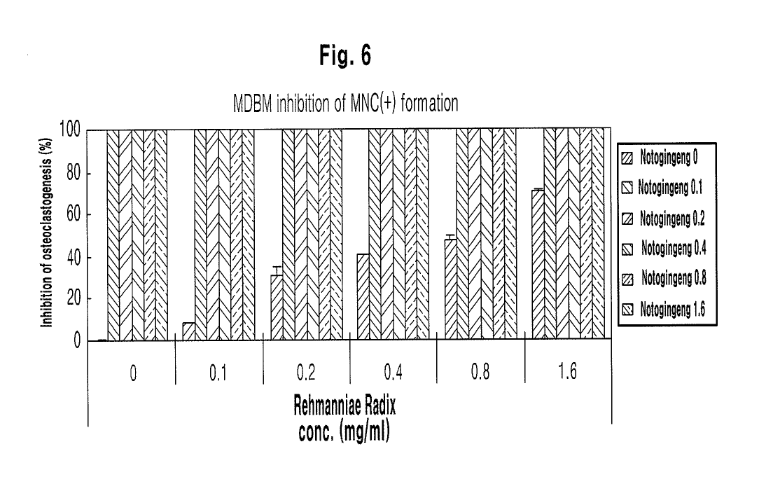

The treatment of the presumable osteoclast TRAP(+) multinucleate cells with the extract of the present invention results in greatly suppressing the formation of TRAP(+) multinucleate cells and the activity of osteoclast cells as the concentration of the extract increases. Particularly, a mixture extract of Rehmanniae Radix Preparata and Panax notoginseng (Burk.) F. H. Chen has a significant inhibitory effect on the formation of TRAP(+) multinucleate and the activity of osteoclasts (FIGS. 6-9).

Taken together, the results indicate that the extract according to the present invention has the function of suppressing the secretion of TNF-α, promoting the secretion of OPG and the proliferation of osteoblast cells, and inhibiting the formation of the presumable osteoclast TRAP(+) multinucleated cells and the activity of osteoclasts. Therefore, the extract according to the present invention can be used for the prevention and treatment of periodontal diseases, such as gingivitis and periodontitis.

In addition to the active ingredient, the pharmaceutical composition for the treatment and prevention of periodontal diseases in accordance with the present invention may include at least one pharmaceutically acceptable carrier. Examples of the pharmaceutically acceptable carrier include saline solution, sterile water, Ringer's solution, buffered saline solution, dextrose solution, maltodextrin solution, glycerol, ethanol, and a mixture of two or more thereof. If necessary, the composition may further include other conventional additives, such as antioxidants, buffers, and bacteriostatic agents. Also, the composition may additionally include diluents, dispersants, surfactants, binders and lubricants in order to be formulated into injection formulations, such as aqueous solution, suspension and emulsion, pills, capsules, granules or tablets. Furthermore, the composition may be preferably formulated depending on its components or purposes, using a suitable method known in the art, for example, the method described in Remington's Pharmaceutical Science (latest edition), Mack Publishing Company, Easton Pa.

The administration route for the pharmaceutical composition of the present invention is not particularly restricted, but, according to the intended use, the composition may be administered orally or via parenteral routes, for example, intravenous, subcutaneous, intraperitoneal or topical. The specific therapeutically effective dose level for any particular patient may vary depending on a variety of factors, including the patient's weight, age, gender, general health status and diet, the time of administration, route of administration, rate of excretion of the composition, and severity of the illness. The composition may be administered in a daily dosage ranging from about 0.1 to 1000 mg/kg, and preferably 0.1 to 500 mg/kg. The daily dosage can be given in a single dose or in several divided doses.

For the effective treatment and prevention of periodontal diseases, the pharmaceutical composition may be preferably formulated into an oral preparation. The oral preparation is not particularly limited, but may be in a general form. Examples of the oral preparation include toothpaste, mouthwash and mouthrinse. Depending on dosage forms, the oral preparation may include various base ingredients and additives necessary to for the formulation thereof. The kinds and amounts of the ingredients and additives may be readily selected by those skilled in the art. For example, when the oral preparation is toothpaste, it may include an abrasive, a wetting agent, a foaming agent, a binder, a sweetener, a pH modifier, a preservative, an medicinally effective ingredient, a bleaching agent, a colorant, a solvent, etc.

The present invention also provides a health food composition for the prevention and treatment of periodontal diseases, comprising an extract from Rehmanniae Radix Preparata, Panax notoginseng (Burk.) F. H. Chen, or a mixture thereof.

The extract from Rehmanniae Radix Preparata, Panax notoginseng (Burk.) F. H. Chen or a mixture thereof can be prepared through a conventional method such as cold precipitation, hot precipitation, heating, etc. Preferably, Rehmanniae Radix Preparata, Panax notoginseng (Burk.) F. H. Chen or a mixture thereof is treated with water, alcohol or a mixture thereof. 8 weight parts of Panax notoginseng (Burk.) F. H. Chen is preferably used per weight part of Rehmanniae Radix Preparata. The alcohol is preferably methanol or ethanol. More preferably, Rehmanniae Radix Preparata, Panax notoginseng (Burk.) F. H. Chen or a mixture thereof is immersed in methanol or ethanol at 70-80° C. for 3-5 hrs. The repetition of this procedure may increase the efficiency of extraction. The concentration may be carried out under conditions of 20-40° C. in a vacuum, but is not limited thereto. As for the individual extracts from Rehmanniae Radix Preparata or Panax notoginseng (Burk.) F. H. Chen, they may be mixed with each other at a weight ratio of 1-16:16-1.

With the aim of alleviating periodontal diseases, the extract according to the present invention may be added to health food. As a food additive, the herbal mixture extract may be properly used alone or in combination with other food ingredients according to a conventional method. The amount of the herbal mixture extract may vary depending on the purpose thereof (prevention, health improvement or therapeutic treatment). Generally, when used for the preparation of foods or beverages, the extract according to the present invention may be added in an amount of 100 wt % or less based on the weight of the material and preferably in an amount of 50 wt % or less. In the case where the extract is applied to health foods which are designed to be taken habitually, its content may be below the above-mentioned range. However, the extract has no problems of safety to the body and thus can be used in an amount exceeding the range.

No particular limitations are imposed on the kind of the foods to which the extract can be applied. Examples of the foods include meat, sausages, bread, chocolate, candies, snacks, confectionaries, pizza, ramen, noodles, dairy products, soups, beverages, drinks, alcoholic beverages, vitamin tablets, etc. and are not limited thereto. All usually accepted health foods may contain the extract according to the present invention.

Like currently available teas, the health food composition of the present invention may further contain various fragrant or natural carbohydrates. Examples of such natural carbohydrates include monosaccharides, such as glucose and fructose, disaccharides such as maltose and sucrose, polysaccharides such as dextrin and cyclodextrin, and sugar alcohols such as xylitol, sorbitol and erythritol. Also, sweeteners, e.g., natural sweeteners such as thaumatin and a stevia extract, or synthetic sweeteners such as saccharin and aspartame, may be added to the health food to which the extract of the present invention is applied. The natural carbohydrate may be used in an amount of about 0.1˜20 g based on 100 ml of the composition of the present invention, and preferably in an amount of about 1˜10 g. In addition, the composition of the present invention may contain various nutrients, vitamins, minerals (electrolytes), flavors, colorants, pectic acid or salts thereof, alginic acid or salts thereof, organic acids, protective colloidal thickeners, pH modifiers, stabilizers, antiseptics, glycerin, alcohols, and carbonating agents used in carbonated beverages. Moreover, the composition of the present invention can contain fruit flesh for preparing natural fruit juices, fruit beverages and vegetable beverages. These ingredients may be used individually or in combination. The ratio of these additives is not important, but is generally selected in a range of 0.05 to 50 parts by weight per 100 parts by weight of the composition of the present invention.

### MODE FOR INVENTION

A better understanding of the present invention may be obtained through the following examples which are set forth to illustrate, but are not to be construed as the limit of the present invention.

### Example 1

### Preparation of Herbal Mixture Extract from Rehmanniae Radix Preparata and Panax notoginseng (Burk.) F. H. Chen

### Example 1-1

### Preparation of Herbal Mixture Extract from Rehmanniae Radix Preparata and Panax notoginseng (Burk.) F. H. Chen with Water

Rehmanniae Radix Preparata and Panax notoginseng (Burk.) F. H. Chen were purchased from a medicinal herb store. After the examination of the herbs Panax notoginseng (Burk.) F. H. Chen and Rehmanniae Radix Preparata for impurities, only clean herbs were selected for use in the following experiment. Panax notoginseng (Burk.) F. H. Chen was finely chopped and Rehmanniae Radix Preparata [symbol] was sliced into pieces 1˜2 cm in size. 200 g of a mixture of 8:1 Panax notoginseng (Burk.) F. H. Chen: Rehmanniae Radix Preparata was placed in a 3 L flask, followed by three rounds of extraction with 2,000 ml of distilled water at 100° C. for 8 hours under a flux condition. After being pooled, the resulting solution was filtered and concentrated into a volume of 500 ml at 40° C. or lower in a vacuum using an evaporator. Following centrifugation (3,000 rpm, 20 min), the supernatant was freeze-dried into powder for use in the experiment (yield 62%).

### Example 1-2

### Preparation of Herbal Mixture Extract from Rehmanniae Radix Preparata and Panax notoginseng (Burk.) F. H. Chen with Ethanol

The same procedure as in Example 1-1 was performed with the exception that ethanol was used instead of water.

### Example 1-3

### Preparation of Herbal Mixture Extract from Rehmanniae Radix Preparata and Panax notoginseng (Burk.) F. H. Chen with 75% Ethanol

The same procedure as in Example 1-1 was performed with the exception that a mixture of 25% water and 75% ethanol was used instead of water.

### Example 2

### Preparation of Extract from Panax notoginseng (Burk.) F. H. Chen

100 g of Panax notoginseng (Burk.) F. H. Chen was finely pulverized and placed, or its powder was placed in a 2 L extractor and added with 10 weight parts of distilled water, followed by hot extraction at 95˜100° C. for 8 hours. The resulting solution was filtered through a suction filter and centrifuged (3,000 rpm, 20 min). After the supernatant was filtered, the filtrate was concentrated at 40° C. using a vacuum evaporator and freeze-dried into powder for use in the experiment (yield 33%).

### Example 3

### Preparation of Extract from Rehmanniae Radix Preparata

100 g of Rehmanniae Radix Preparata was placed in a 2 L extractor and added with 10 weight parts of distilled water, followed by hot extraction at 95˜100° C. for 8 hours. The resulting solution was filtered through a suction filter, concentrated at 40° C. using a vacuum evaporator and stored in a refrigerator until use (yield 45%).

### Experimental Example 1

### Suppression of TNF-α Secretion

The extract according to the present invention was assayed for inhibitory activity against the release of TNF-α from the human monocytic cell line THP-1.

The human monocytic cell line THP-1 (ATCC No. TIB-202) was purchased from ATCC (Rockville, USA) and cultured in an RPMI 1640 medium (Gibco BRL, USA) supplemented with 10% FBS (Fetal bovine serum) before use in experiments. The cells were placed at a density of 5×10⁵ cells/ml into 96-well plates and stimulated with LPS to secrete TNF-α.

To experimental groups, respective extracts from Rehmanniae Radix Preparata and Panax notoginseng (Burk.) F. H. Chen were added, in combination, in an amount of 0˜1.6 mg/ml, along with LPS. Alternatively, the experimental groups were treated with was 0˜1.6 mg/ml of the mixture extract, together with LPS. 24 hours after the treatment, the culture media were analyzed for TNF-α level using ELISA, and the results are summarized in FIGS. 1 and 2 and Table 1.

TABLE 1

| Rehmanniae Radix | Panax notoginseng (mg/ml) | | | | | |
|---|---|---|---|---|---|---|
| Preparata (mg/ml) | 0 | 0.1 | 0.2 | 0.4 | 0.8 | 1.6 |
| 0 | 107.93 ± 1.58 | 94.16 ± 2.32 | 81.89 ± 4.14 | 71.26 ± 3.44 | 55.39 ± 0.40 | 45.21 ± 1.82 |
| 0.1 | 98.35 ± 6.03 | 83.23 ± 3.14 | 70.36 ± 3.54 | 63.47 ± 3.58 | 60.48 ± 6.59 | 46.56 ± 6.14 |
| 0.2 | 93.41 ± 1.44 | 79.64 ± 2.87 | 70.96 ± 1.44 | 60.33 ± 3.48 | 52.25 ± 2.60 | 42.96 ± 1.43 |
| 0.4 | 111.09 ± 3.78 | 107.09 ± 5.73 | 93.22 ± 2.14 | 91.22 ± 3.75 | 68.10 ± 3.13 | 55.16 ± 1.26 |
| 0.8 | 100.77 ± 3.15 | 92.60 ± 3.93 | 77.97 ± 1.34 | 81.66 ± 4.84 | 57.47 ± 1.47 | 48.07 ± 0.71 |
| 1.6 | 103.85 ± 1.08 | 87.67 ± 4.75 | 85.05 ± 3.95 | 69.49 ± 2.07 | 61.79 ± 5.63 | 47.92 ± 0.82 |

As seen in FIG. 1 and Table 1, the cells which were treated with various combinations of 0˜1.6 mg/ml of respective extracts of Rehmanniae Radix Preparata and Panax notoginseng (Burk.) F. H. Chen were observed to be significantly restrained from TNF-α release in a dose-dependent manner as compared to the control.

Also, as seen in FIG. 2, treatment with the herbal mixture extract according to the present invention was found to significantly inhibit the secretion of TNF-α in a dose-dependent manner as compared to the control not treated therewith.

Therefore, the extracts according to the present invention effectively inhibit the secretion of TNF-α.

### Experimental Example 2

### Assay for Osteoblast Proliferation

The extracts according to the present invention were analyzed for effect on the proliferation of osteoblast cells.

For use as osteoblastic cells, murine calvarial MC3T3-E1 (ATCC No. CRL-2593) cells were purchased from ATCC (Rockville, USA) and cultured in DMEM (Gibco BRL, USA) supplemented with 10% FBS (Fetal bovine serum).

The osteoblastic cells were plated at a density of 20,000 cells per well onto 24-well plates and incubated for 48 hours in a 5% CO₂ incubator. Then, the culture medium was replaced with fresh media to which respective extracts from Rehmanniae Radix Preparata and Panax notoginseng (Burk.) F. H. Chen were added, in combination, in an amount of 0˜1.6 mg/ml, followed by incubation for an additional 48 hours. Following the aspiration of the culture media, the cells were treated with trypsin-EDTA and counted with a hemacytometer. The results are graphed in FIG. 3.

As seen in FIG. 3, the treatment with the extracts according to the present invention was observed to promote the proliferation of the cells by up to 20% according to concentration. The cells which were treated with various combinations of 0˜1.6 mg/ml of respective extracts from Rehmanniae Radix Preparata and Panax notoginseng (Burk.) F. H. Chen were observed to increase in cell number compared to the control.

### Experimental Example 3

### Assay for Expression of OPG (Osteoprotegerin)

The human osteosarcoma MG-63 cells (ATCC No. CRL-1427) were cultured to confluency in 60 mm tissue culture dishes and incubated in 2 ml of DMEM containing various combinations of 0˜1.6 mg/ml of respective extracts from Rehmanniae Radix Preparata and Panax notoginseng (Burk.) F. H. Chen or 0˜1.6 mg/ml of the herbal mixture extract for 24 hours. Thereafter, the culture media were analyzed for OPG level using an OPG-ELISA kit (Oscotec Inc.). The results are given in FIGS. 4 and 5.

As seen in FIG. 4, the expression of OPG, inhibitory protein of the formation of osteoclast cells, in osteoblasts was observed to be further promoted when the cells were treated with 0.1˜1.6 mg/ml of the herbal mixture extract from Rehmanniae Radix Preparata and Panax notoginseng (Burk.) F. H. Chen than with respective extracts. The OPG expression level of the groups treated with the herbal mixture extract was increased in a dose-dependent manner up to 300% compared to that of the groups treated with individual extracts.

Also, as seen in FIG. 5, the treatment with a mixture extract from Rehmanniae Radix Preparata and Panax notoginseng (Burk.) F. H. Chen was found to significantly increase the expression of OPG compared to the control.

### Experimental Example 4

### Inhibition of Formation of TRAP(+) Multinucleated Cells

After male mice 7˜9 weeks old were sacrificed by cervical dislocation, the femur and the tibia were aseptically excised and cleared of soft tissues. Then, the iliac bone was cut at opposite ends, and the bone marrow was pulled out by injecting 1 ml of an enzyme solution containing 0.1% collagenase (Gibco), 0.05% trypsin and 0.5 mM EDTA (Gibco) into the bone marrow cavity of one end through a 26G needle. Bone marrow cells were obtained by stirring the bone marrow for 30 min and pre-cultured in α-MEM supplemented with 10% FBS for 24 hours to obtain non-adherent cells which are osteoclastic progenitor cells. They were plated at a density of 2><10⁵ cells per well and cultured.

Of the cultured cells, adherent cells were washed with PBS and fixed for 5 min with citrate-acetate-formaldehyde, followed by TRAP staining by incubation at 37° C. in an acetate buffer (pH 5.0) containing naphthol AS-BI phosphate, Fast Garnet GBC buffer and 7 mM tartrate buffer (pH 5) for 1 hour. TRAP(+) multinucleated cells with three or more nuclei were regarded osteoclastic cells.

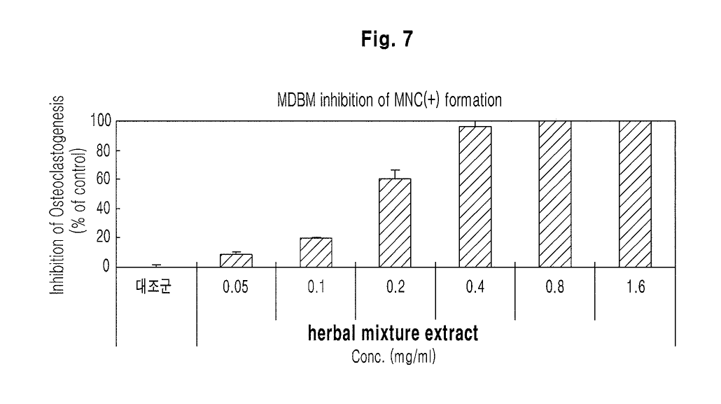

In order to induce the differentiation of osteoclasts, the bone marrow in which osteoclastic progenitor cells are present were utilized. The TRAP-positive multinucleated cells were regarded as osteoclastic cells which were then cultured in the presence of 0˜1.6 mg/ml of individual herbal extracts or 0˜1.6 mg/ml of the herbal mixture extract, followed by monitoring the counts of the TRAP-positive multinucleated cells. The results are graphed in FIGS. 6 and 7. As seen in FIGS. 6 and 7, the groups treated with the extracts according to the present invention were observed to significantly decrease in TRAP(+) multinucleated cell count. From these results, it is demonstrated that the extracts according to the present invention are useful in the inhibition of the formation of osteoclastic cells.

### Experimental Example 5

### Inhibition of Osteoclast Activity

For monitoring the growth and activity of osteoclastic cells, osteoclastic progenitor cells were cultured in carbonated calcium phosphate-coated plates (OAAS, OCT Inc.) to observe the activity and resorption of the osteoclastic label TRAP.

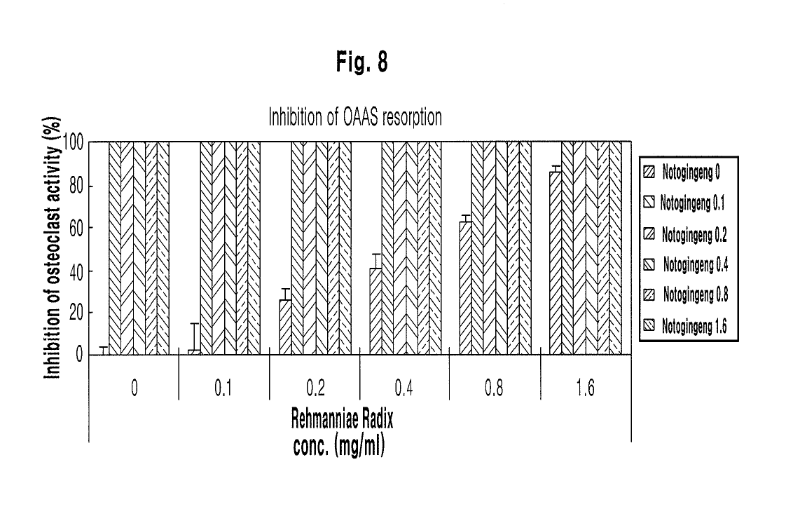

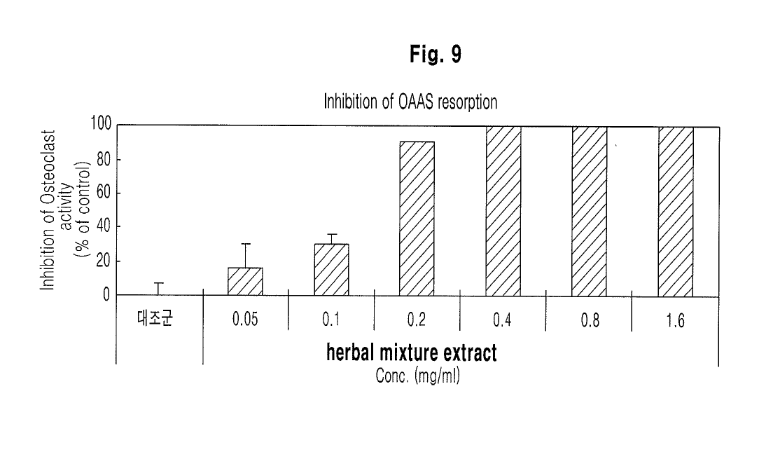

The culture medium was removed from the osteoclastic progenitor cells separated and cultured in Experimental Example 4. The OAAS plates were washed with distilled water to remove adherent cells, incubated for 5 min in a 5% sodium hypochlorite solution, and then washed with distilled water before the observation of the absorption regions using Image Pro plus. That is, in order to examine the activity of osteoclasts, which are responsible for bone resorption in bone tissues, osteoclastic progenitor cells were cultured in plates coated with calcium and phosphate, which were designed to make a condition similar to the mineral part of the bone tissue, in the presence of the extracts according to the present invention. Changes in absorption area were monitored, and the results are shown in FIGS. 8 and 9.

As seen in FIGS. 8 and 9, the extracts according to the present invention were observed to significantly inhibit the activity and resorption of osteoclasts as measured with calcium-phosphate-coated plates for culturing osteoclastic progenitor cells.

### Experimental Example 6

### Effect on Proliferation of Gingival Fibroblast and Periodontal Ligament Cells

The extracts according to the present invention were assayed for their effect on the proliferation of human periodontal ligament cells and gingival fibroblasts.

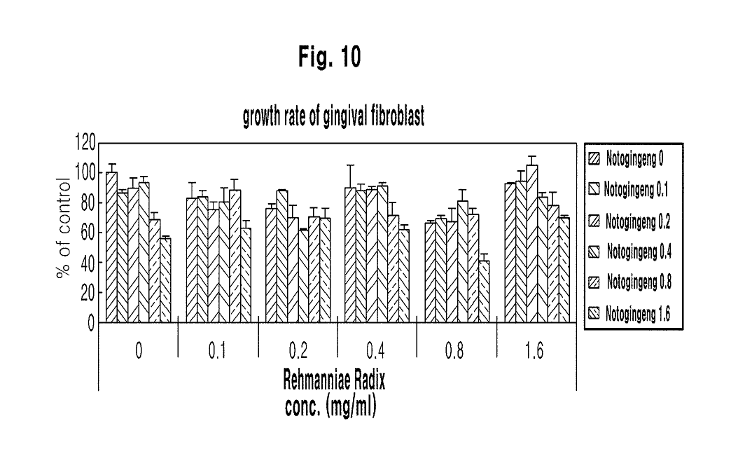

The periodontal ligament of a premolar drawn with the aim of orthodontia from a healthy human was aseptically scraped with a curette and placed in a culture dish, followed by incubation in DMEM (Gibco BRL, USA) supplemented with 10% FBS (fetal bovine serum). A portion of the gums of the drawn premolar was separated and cultured in the same manner to obtain gingival fibroblasts. During culture growth, cell outgrowths from explants were examined with a microscope. After the formation of cell colonies, respective tissue specimens were removed therefrom, allowed to grow in single layers, and subcultured until use in experiments. Each of the human periodontal ligament cells and gingival fibroblasts was plated at a density of 20,000 cells per well onto 24-well plates and incubated for 48 hours in a 5% CO₂ incubator, after which the cells were treated with 0˜1.6 mg/ml of the individual extracts or 0˜1.6 mg/ml of the mixture extract and further incubated for an additional 48 hours. After the removal of the culture media, trypsin-EDTA was added and the cells were counted with a hemacytometer. The results are graphed in FIGS. 10, 11 and 12.

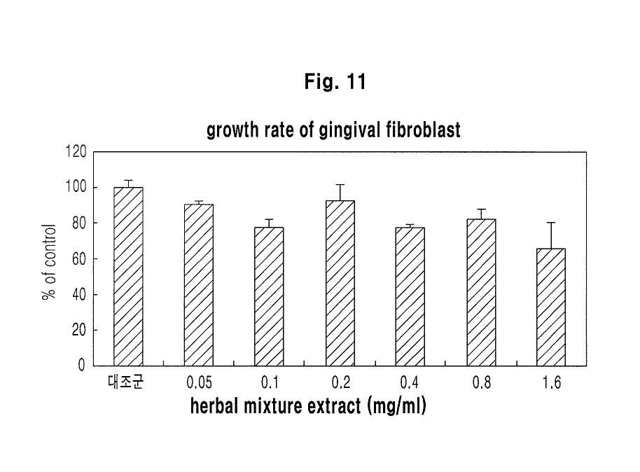

As seen in FIGS. 10 and 11, the gingival fibroblasts were observed to proliferate upon treatment with the herbal mixture extract at a level as low as or less than upon treatment with the individual extracts.

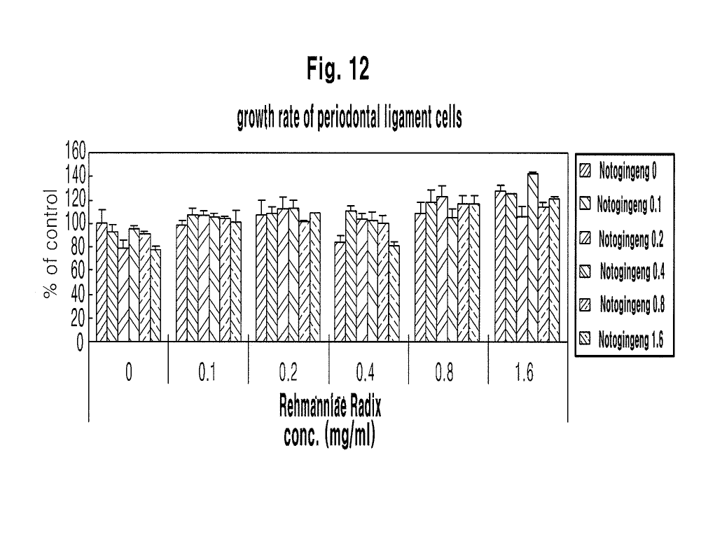

As seen in FIG. 12, the proliferation of the periodontal ligament cells was further promoted by up to 40% upon treatment with the herbal mixture extract as compared to treatment with the individual extracts from Rehmanniae Radix Preparata or Panax notoginseng (Burk.) F. H. Chen.

Therefore, the extracts according to the present invention can effectively promote the proliferation of periodontal ligament cells, which play a critical role in the regeneration of the periodontal tissue, without significant affection on the proliferation of gingival fibroblasts.

### Experimental Example 7

### Therapeutic Efficacy in Animal Model with Periodontal Diseases

Periodontal diseases are chronic diseases caused by complex factors. Of them, the most important is bacterial contribution. To the surface of a tooth is attached a bacterial biofilm which is a complex aggregation of microorganisms in combination with salivary proteins and foods. Patients with periodontal diseases, most of which are defective in the function of immune defense cells, are unable to resist various products from the bacteria and suffer from severe tissue destruction.

In an experimental rat model, a silk thread was tied to a tooth to spontaneously generate plaque and calculus thereat, which then caused inflammation, resulting in the resorption of the alveolar bone.

A rat molar was tied up with a ligature to induce periodontal disease and the rats were administered with the extracts according to the present invention. The maxillary bone including the tooth was excised and observed to examine the effect of the extracts on the prevention and treatment of the periodontal disease.

After rats were generally anesthetized by abdominally injecting a mixture of ketamine (100 mg/kg) and xylazine (10 mg/kg), a second molar of the inferior maxilla was tied up with a silk ligature (4/0) to induce periodontal disease.

A sham and a negative control were orally administered with solid foodstuff and 5 ml/kg of water while experimental groups were orally administered with the extracts of the present invention (100 mg/kg) as well, at the same time for 4 weeks.

After completion of the oral administration, the inferior maxilla was excised from the rats anesthetized with CO₂ and fixed for 2 hours with a Bouin's solution. Afterwards, it was subjected to decalcification with 5% nitric acid for 72 hours to remove minerals including calcium to soften the tissue to the degree suitable for tissue section. Subsequently, the teeth were washed with flowing water for 12 hours and dehydrated with 70%, 80%, 90% and 100% alcohol in that order three times for 2 hours per each concentration, followed by three rounds of substitution with xylene for 2 hours per round. The samples were embedded in paraffin by three rounds of treatment with liquid paraffin for 2 hours per round.

After the construction of paraffin blocks, specimens were made by slicing the blocks into a thickness of 5 μm using a rotary microtome, attaching the slice onto a slide glass and drying using a slide warmer (40±3° C.). The completely dried slides were deprived of paraffin using xylene, dehydrated with alcohol, and stained with hematoxylin-eosin or trichrome. Thereafter, the stained tissues were encapsulated by being passed again through alcohol and xylene, followed by drying overnight at 60° C. in an incubator. They were observed under an optical microscope and photographed.

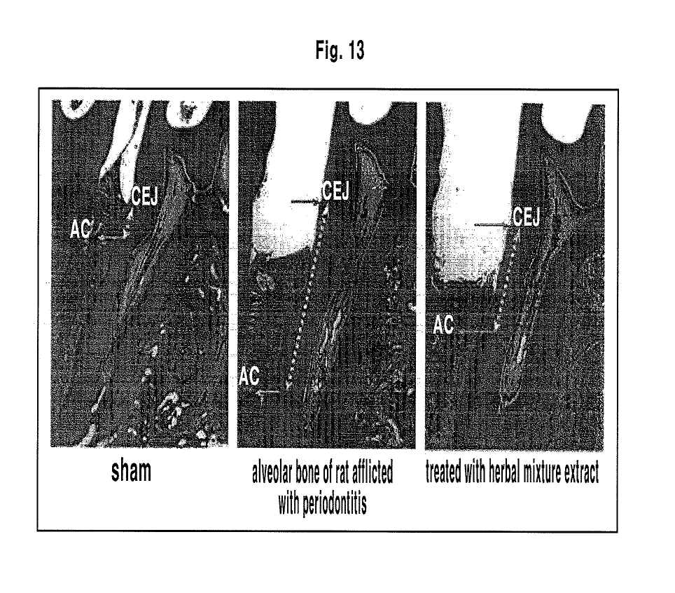

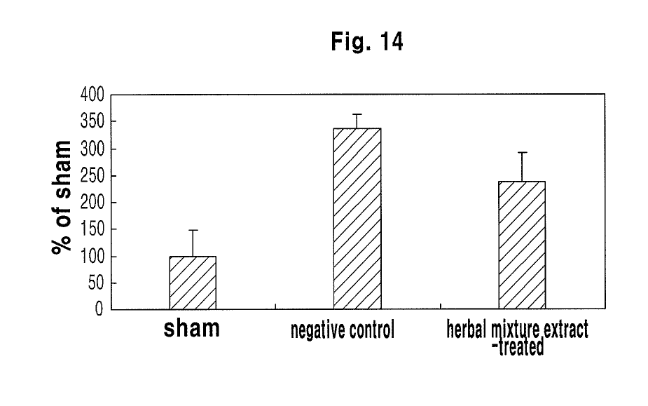

Using an image analysis system (Image-pro plus; version 3.0), the photographs were assayed for the distance between the alveolar bone crest (AC) and the cemento-enamel junction (CEJ). The inhibitory effect of the herbal mixture extract from Rehmanniae Radix Preparata and Panax notoginseng (Burk.) F. H. Chen was determined by comparing the distance between AC and CEJ of the experimental groups with that of the sham or the negative control. The results are shown in FIGS. 13 and 14.

Compared to the sham, the negative control that was fed only with water after periodontal disease induction, as shown in FIGS. 13 and 14, increased three fold in bone loss, but the groups administered with the herbal mixture extract from Rehmanniae Radix Preparata and Panax notoginseng (Burk.) F. H. Chen increased only twofold.

As demonstrated in the rats with periodontal diseases, the extracts according to the present invention are useful in the treatment and prevention of periodontal diseases.

Below, a description will be given of formulation examples of the composition according to the invention.

### Formulation Example 1

### Pharmaceutical Preparations

1. Preparation of Powder

| | |
|---|---|
| Extract according to the present invention | 2 g |
| Lactose | 1 g |

These ingredients were admixed and loaded into an airtight sac to prepare a powder form.

2. Preparation of Tablet

| | |
|---|---|
| Extract according to the present invention | 100 mg |
| Corn starch | 100 mg |
| Magnesium Stearate | 2 mg |

These ingredients were admixed and tabletted according to a typical method to produce tablets.

3. Preparation of Capsule

| | |
|---|---|
| Extract according to the present invention | 100 mg |
| Corn starch | 100 mg |
| Lactose | 100 mg |
| Magnesium stearate | 2 mg |

These ingredients were admixed and loaded into gelatin capsules according to a typical method to produce capsules.

4. Preparation of Mouthwash

| Extract according to the present invention | 0.01~1.0 | g |
|---|---|---|
| Xylitol | 5~10 | g |
| Ethyl alcohol | 5~15 | g |
| Sorbitol | 5~15 | g |
| Sodium saccharin | 10~100 | mg |
| Sodium monofluorophosphate | 500~1000 | mg |
| Sodium laurylsulfate | 100~200 | mg |
| Polysorbate 20 | 100~1000 | mg |
| Peppermint flavor | 10~100 | mg |
| Sodium benzoate | 10~100 | g |
| Pure water | suitable amount | |
| color | suitable amount | |
| Total | 100 | g |

These ingredients were admixed according to a typical method to produce mouthwash useful in the prevention and treatment of periodontitis.

### Formulation Example 2

### Preparation of Food

Foods containing the extract according to the present invention were prepared as follows.

1. Preparation of Seasoning

A health-improving seasoning containing the extract according to the present invention in an amount of 20˜95 wt % was prepared.

2. Preparation of Tomato Ketchup and Source

Health-improving tomato ketchup or source was prepared by adding the extract according to the present invention in an amount of 0.2˜1.0 wt % to typical tomato ketchup or source.

3. Preparation of Flour Food

A flour mixture containing 0.5˜5.0 wt % of the extract according to the present invention was used to make breads, cakes, cookies and noodles.

4. Preparation of Soup and Gravies

The extract according to the present invention was added in an amount of 0.1˜5.0 wt % to typical soup or gravies to prepare health-improving soup or gravies for meat processed products or noodles.

5. Preparation of Ground Beef

The extract according to the present invention was added in an amount of 10 wt % to typical ground beef to prepare health-improving ground beef.

6. Preparation of Dairy Products

Milk containing 5˜10 wt % of the extract according to the present invention was used to prepare various dairy products such as butter and ice cream.

7. Preparation of Zen Food

Unmilled rice, barley, glutinous rice, and unshelled adlay were pregelatinized using a typical method, dried and roasted before grinding into powder with a particle size of 60 meshes.

Black soybean, black sesame and wild sesame were steamed according to a typical method, dried and roasted before grinding into powder with a particle size of 60 meshes.

The extract according to the present invention was concentrated in a vacuum using a vacuum concentrator and dried in a convection oven, followed by grinding into powder with a particle size of 60 meshes.

The powders made of the grains, the seeds, and the extract according to the present invention were formulated at the following ratios to yield a zen food.

Grains (unmilled rice 30 wt %, unshelled adlay 15 wt %, barley 20 wt %),

Seeds (wild sesame 7 wt %, black soybean 8 wt %, black sesame 7 wt %),

Dry powder of the extract according to the present invention (3 wt %),

Ganoderma lucidum (0.5 wt %),

Foxglove (0.5 wt %)

### Formulation Example 3

### Preparation of Beverage

1. Preparation of Carbonated Beverage

A mixture containing 5˜10% of sugar, 0.05˜0.3% of citric acid, 0.005˜0.02% of caramel and 0.1˜1% of vitamin C was admixed with 79˜94% of pure water to give syrup which was then sterilized at 85˜98° C. for 20˜180 sec. The sterilized syrup was mixed at a ratio of 1:4 with cold water, followed by injecting carbon dioxide 0.5˜0.82% of carbon dioxide to afford a carbonated beverage containing the extract according to the present invention.

2. Preparation of Health Beverage

Liquid fructose (0.5%), oligosaccharide (2%), sugar (2%), salt (0.5%) and water (75%) were homogeneously formulated, along with the extract according to the present invention and the formulation was subjected to pasteurization and loaded into a bottle, such as glass bottle, PET bottle, etc.

3. Preparation of Vegetable Juice

5 g of the extract according to the present invention was added to 1,000 ml of typical tomato or carrot juice to give medicinal vegetable juice.

4. Preparation of Fruit Juice

1 g of the extract according to the present invention was added to 1,000 ml of typical apple or grape juice to give medicinal fruit juice.

1-18. (canceled)

19. A method for treating a periodontal disease, comprising administering one or more of an extract from Rehmanniae Radix Preparata, an extract from Panax notoginseng (Burk.) F. H. Chen, a mixture of the extracts, or an extract from a mixture of Rehmanniae Radix Preparata and Panax notoginseng (Burk.) F. H. Chen into a subject who is in need thereof.

20. The method according to claim 19, wherein the mixture of the extract from Rehmanniae Radix Preparata and the extract from Panax notoginseng (Burk.) F. H. Chen has a weight ratio of 1:16-16:1 of the extract from Rehmanniae Radix Preparata: the extract from Panax notoginseng (Burk.) F. H. Chen.

21. The method according to claim 19, wherein the mixture of Rehmanniae Radix Preparata and Panax notoginseng (Burk.) F. H. Chen has a weight ratio of 1:8 of Rehmanniae Radix Preparata and Panax notoginseng (Burk.) F. H. Chen.

22. The method according to claim 19, wherein the extract from Rehmanniae Radix Preparata and the extract from Panax notoginseng (Burk.) F. H. Chen, or the extract from the mixture of Rehmanniae Radix Preparata and Panax notoginseng (Burk.) F. H. Chen is prepared by using water, alcohol or a mixture of alcohol and water as an extraction solvent.

23. The method according to claim 22, wherein the alcohol is methanol or ethanol.

24. The method according to claim 19, wherein the extract or the mixture of the extracts has an activity to suppress secretion of tumor necrosis factor.

25. The method according to claim 19, wherein the extract or the mixture of the extracts has an activity to promote secretion of osteoprotegerin.

26. The method according to claim 19, wherein the periodontal disease is gingivitis.

27. The method according to claim 19, wherein the periodontal disease is periodontitis.

28. The method according to claim 19, wherein the extract or the mixture of the extracts is orally administered.

29. The method according to claim 19, wherein the therapeutically effective amount is in the range of 0.1 to 1000 mg/kg body weight/day.

30. A health food composition for the alleviation of a periodontal disease, comprising one or more of an extract from Rehmanniae Radix Preparata, an extract from Panax notoginseng (Burk.) F. H. Chen, a mixture of the extracts, or an extract from a mixture of Rehmanniae Radix Preparata and Panax notoginseng (Burk.) F. H. Chen as an active ingredient.

31. The health food composition according to claim 30, wherein the mixture of the extract from Rehmanniae Radix Preparata and the extract from Panax notoginseng (Burk.) F. H. Chen has a weight ratio of 1:16-16:1 of the extract from Rehmanniae Radix Preparata: the extract from Panax notoginseng (Burk.) F. H. Chen.

32. The health food composition according to claim 30, wherein the mixture of Rehmanniae Radix Preparata and Panax notoginseng (Burk.) F. H. Chen has a weight ratio of 1:8 of Rehmanniae Radix Preparata and Panax notoginseng (Burk.) F. H. Chen.

33. The health food composition according to claim 30, wherein the extract from Rehmanniae Radix Preparata and the extract from Panax notoginseng (Burk.) F. H. Chen, or the extract from the mixture of Rehmanniae Radix Preparata and Panax notoginseng (Burk.) F. H. Chen is prepared by using water, alcohol or a mixture of alcohol and water as an extraction solvent.

34. The health food composition according to claim 33, wherein the alcohol is methanol or ethanol.

35. The health food composition according to claim 30, wherein the periodontal disease is gingivitis.

36. The health food composition according to claim 31, wherein the periodontal disease is periodontitis.
